(19) United States
(12) Patent Application Publication
(10) Pub. No.: US 2018/0126075 A1
(43) Pub. Date: May 10, 2018

(54) SYSTEM, DEVICE AND METHOD FOR AUTOMATED TREATMENT OF SYMPTOMS ASSOCIATED WITH NERVE GAS EXPOSURE

(71) Applicant: ARKHAM ENTERPRISES, LLC, Dover, DE (US)

(72) Inventors: Irving D. Kaplan, Dedham, MA (US); Edward J. Holupka, Medway, MA (US)

(21) Appl. No.: 15/860,139

(22) Filed: Jan. 2, 2018

Related U.S. Application Data
(63) Continuation-in-part of application No. 15/695,618, filed on Sep. 5, 2017.
(60) Provisional application No. 62/411,069, filed on Oct. 21, 2016.

(51) Int. Cl. A61M 5/172; A61K 31/46; A61K 31/44; A61K 31/5513; A61K 9/00; A61M 5/14

(52) U.S. Cl. CPC A61M 5/1723; A61M 2205/054; A61K 31/44; A61K 31/5513; A61K 9/0019; A61M 5/1407; A61M 2205/50; A61M 2205/502; A61M 2205/18; A61M 2205/3303; A61M 2230/20; A61M 2205/581; A61M 2205/583; A61M 2205/582; A61M 2205/3584; A61M 2205/3592; A61M 2205/505; A61M 2205/80; A61K 31/46

(57) ABSTRACT

Systems and methods for treating nerve agent exposure in a person are disclosed. Specifically, systems and methods for detecting with sensor(s) the presence of a nerve gas in the vicinity of a person and/or symptoms in a person as a result of exposure to nerve gas, followed by actuation of an alarm and automatic initiation of a programmed injection sequence comprising at least one injection of a nerve gas antidote comprising at least one of atropine, an anticholinesterase reactivator such as 2-PAM and an anti-convulsant such as diazepam.

### CROSS-REFERENCE TO RELATED APPLICATIONS

This application is a continuation-in-part of U.S. application Ser. No. 15/695,618, filed Sep. 5, 2017 and entitled SYSTEM AND METHODS FOR OVERDOSE MITIGATION, claiming the benefit of U.S. Provisional Application Ser. No. 62/411,069, filed Oct. 21, 2016, and entitled SYSTEMS AND METHODS FOR OVERDOSE MITIGATION, the entirety of each of which is hereby incorporated by reference.

### STATEMENT REGARDING FEDERALLY SPONSORED RESEARCH OR DEVELOPMENT

Not Applicable

### FIELD

The present disclosure relates to systems and methods for preventing or mitigating the effects of nerve agent exposure. More specifically, disclosed embodiments relate to preventing death from exposure to nerve agents, such as sarin, by automatic delivery of nerve agent antidotes, e.g., atropine followed by, or in combination with, an acetylcholinesterase reactivator such as 2-PAM, within seconds of detection of the presence of a nerve agent in the immediate environment or detection of symptoms of nerve agent exposure and, in some cases when convulsing is sensed, automatic delivery of a convulsant antidote such as diazepam.

### BACKGROUND

Chemical warfare agents are generally classified by their mechanism of action and include blood agents and nerve agents. Nerve agent examples include without limitation: sarin, VX, tábun, and soman.

Nerve agents inhibit the normal action of acetylcholinesterase, a chemical compound that serves to break down acetylcholine which causes muscular contraction. Thus, nerve agents inhibit the breaking down of acetylcholine resulting in violent muscle contractions and spasms.

A general antidote to exposure to nerve agents consists of a series of two injections: a first injection with an effective amount of atropine, followed by a second injection of an acetylcholinesterase reactivator compound. The atropine works to protect against excess acetylcholine formation occurring as a result of the nerve gas exposure and poisoning. The reactivator restores acetylcholinesterase activity to its normal function. A typical acetylcholinesterase reactivator is pralidoxime (2-PAM), though other compounds may be used.

Because nerve agents act rapidly, it is critical to administer the treatment comprising atropine and the acetylcholinesterase reactivator either just prior to, or immediately following, exposure to the nerve agent.

It is known in the art to inject atropine followed by an injection of 2-PAM to counteract the effects of nerve agents. However, in certain situations, e.g., on the battlefield, the exposed individual, or a companion, must recognize the exposure's imminence or the exposure itself, and then quickly administer the two-injection antidote in order to minimize damage. Typically, the antidote is self-administered into the outer thigh muscle, or the upper outer quadrant of the buttocks and is done through the clothing. In certain cases, where the victim is incapacitated and/or incapable of acting, a companion will administer the antidote injections, again typically through the clothing.

Accuracy, and consistency, in locating the injection site in the recommended area is important, particularly in the case of injection into the buttocks so as to avoid nerves.

The requirement to administer through clothing may adversely affect the quality and efficiency of the antidote's administration by, inter alia, resulting in bending of the injection needle, an insufficient, or sub-optimal, depth of penetration by the injection needle into the subject's flesh. In turn, the efficacy of the antidote may be compromised.

The timing of administration of the antidote, however, is clearly the variable most critical to its success in treating the exposure. Time to administration, therefore, is a factor that can be adversely affected by several variables using the known administration systems and techniques including the delay in administration until: (1) the threat and/or exposure is recognized either by the individual or a companion; (2) the first injection device is manually accessed and manually prepared for injection, followed by manual execution of the atropine injection; and (3) the second injection device is manually accessed and manually prepared for injection, followed by manual execution of the acetylcholinesterase reactivator injection. Some or all of these timing delays may be mitigated. Additionally, the consistency of injection site and injecting through a subject's clothing are variables that may be improved.

Moreover, in certain cases, the subject exposed to nerve gas may go into convulsions. At this stage of the exposure, it is known to administer, using the same manual recognition and injection techniques described above, a convulsant antidote to treat the convulsions. Typically, diazepam is the convulsant antidote used. However, treating a convulsing patient requires optimization of timing (including recognizing the condition, accessing and preparing the injection device and injecting the subject), in addition to injection site optimization and skin proximity.

The present invention is also applicable to prevention or mitigation of symptoms due to accidental narcotics overdosing. Deaths due to accidental narcotics overdoses are a major preventable cause of death. This high rate of overdose deaths occurs in spite of attempts to widely distribute an overdose antidote (e.g., Naloxone), including attempts to widely distribute the antidote to first responders and care givers. The mechanism of death in overdose is generally due to respiratory suppression, which leads to respiratory arrest, hypoxia, and death. Thus, rapid administration of an antidote is critical to prevent deaths due to opioid overdoses.

One example of an antidote is Naloxone, which is an opioid antagonist. It works by binding to opioid receptors in the brain, and can block the effects of narcotics. This leads to a rapid reversal of opiate effects in the central nervous system. Naloxone can be administered intravenously, intranasally, or intramuscularly. An adult dose of Naloxone for an opioid overdose may range from 0.4 to 2 mg/dose and may be repeated every 2 to 3 minutes as needed, up to a maximum cumulative dose (e.g., of around 10 mg). Naloxone is part of overdose kits and has been shown to reduce the number of overdose deaths. However, the overdose victim is unable to self-administer the medication.

The present invention addresses these, inter alia, needs.

### SUMMARY

According to some embodiments, a nerve agent exposure treatment system includes a first injector fluidly communicating with, and coupled to, a first reservoir containing atropine, a second injector fluidly communicating with, and coupled to, a second reservoir containing an acetylcholinesterase reactivator, and a third injector fluidly communicating with, and coupled to, a reservoir containing diazapam. The atropine and acetylcholinesterase reactivator, e.g., 2-PAM, and/or the diazepam may be combined for delivery with a single injector, or two injectors, and may further be combined into a single reservoir, or two reservoirs. The system includes a processor coupled to a user interface, an alarm, and the injector(s). The processor is configured to, in response to detection of the nerve agent, or symptoms of nerve agent exposure, by sensor(s): actuate the alarm; wait a predetermined amount of time (in some embodiments); and actuate the injector(s) unless an input is received from the user interface.

According to some embodiments, an overdose mitigation method includes detecting the presence of a nerve agent in the subject's immediate environment with a nerve agent detection sensor. In addition, or alternatively, a sensor may detect physical symptoms of nerve agent exposure. The method includes, in response to detection of nerve agent in the immediate environment and/or physical symptoms of nerve agent exposure, actuating an alarm, waiting a predetermined amount of time (in some embodiments), and delivering the antidote injection(s) unless an input is received from a user interface device.

### BRIEF DESCRIPTION OF THE DRAWINGS

FIG. 1 is a simplified block diagram showing components of an overdose mitigation system, according to some embodiments.

FIG. 2 illustrates a flow chart of a method, according to some embodiments.

FIG. 3 is a simplified block diagram showing components of a nerve agent exposure treatment system, according to some embodiments.

FIG. 4 illustrates a flow chart of a method, according to some embodiments.

FIG. 5 illustrates a flow chart of a method, according to some embodiments.

FIG. 6 illustrates a block diagram of one embodiment of the present invention.

### DETAILED DESCRIPTION

The following detailed description includes references to the accompanying figures. The example embodiments described herein are not meant to be limiting. Other embodiments may be utilized, and other changes may be made, without departing from the scope of the subject matter presented herein. It will be readily understood that the aspects of the present disclosure, as generally described herein and illustrated in the figures can be arranged, substituted, combined, separated, and designed in a wide variety of different configurations, all of which are contemplated herein.

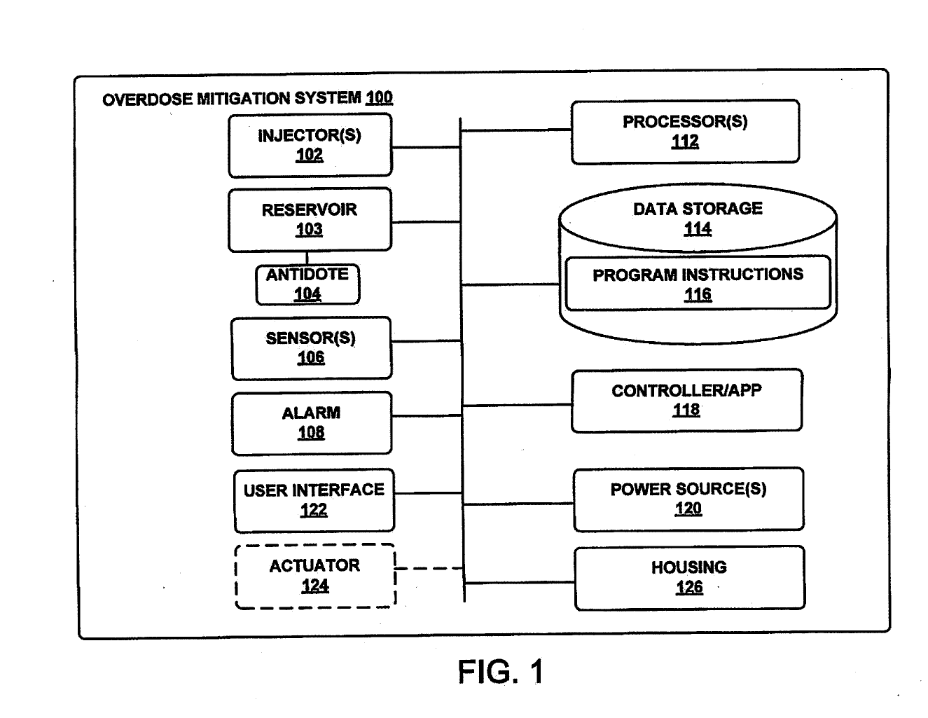

FIG. 1 illustrates a simplified block diagram showing components of an overdose mitigation system, according to some embodiments. Overdose mitigation system 100 includes injector(s) 102, a reservoir 103 for holding an antidote 104 and that is coupled with and/or in fluid communication with the injector(s) 102, sensor(s) 106, an alarm 108, processor(s) 112, data storage 114, program instructions 116, a controller/app 118, power source(s) 120, a user interface 122, actuator 124, and housing 126. The overdose mitigation system 100 is shown for illustration purposes only and may include additional components and/or have one or more components removed without departing from the scope of the disclosure. Further, the various components of overdose mitigation system 100 may be communicatively coupled or otherwise in communication with each other in any manner now known or later developed that enables the components to operate as a system to perform the functionality described herein.

Processor(s) 112 may be a general-purpose processor or a special purpose processor (e.g., digital signal processors, application specific integrated circuits, etc.). The processor(s) 112 can be configured to execute computer-readable program instructions 116 that are stored in the data storage 114 and are executable to cause the overdose mitigation system 100 to perform the functions and features described herein. For instance, the program instructions 116 may be executable to provide functionality of the controller/app 118, where the controller/app 118 may be a smartphone application that is configured to accept a touch input to turn off the alarm and stop an injection. The controller/app 118 may be configured to communicate information as well. For example, the controller/app 118 may be configured to send a text message alert or email to emergency personnel or care givers indicating an overdose has occurred (e.g., after the overdose sensor detects an overdose indicator), that the injector(s) 102 have been used, or anything else.

The data storage 114 may include or take the form of one or more computer-readable storage media that can be read or accessed by processor(s) 112. The one or more computer-readable storage media can include volatile and/or non-volatile storage components, such as optical, magnetic, organic or other memory or disc storage, which can be integrated in whole or in part with processor(s) 112. In some embodiments, the data storage 114 can be implemented using a single physical device (e.g., one optical, magnetic, organic or other memory or disc storage unit), while in other embodiments, the data storage 114 can be implemented using two or more physical devices. Further, in addition to the computer-readable program instructions 116, the data storage 114 may include additional data such as diagnostic data, among other possibilities.

The overdose mitigation system 100 may include one or more sensor(s) 106. For example, sensor(s) 106 may include a pulse oximeter sensor to measure oxygen saturation levels. The pulse oximeter may be connected to the processor(s) 112 and configured to provide an overdose indicator should oxygen saturation levels drop below a predetermined threshold. Sensor(s) 106 may be included in overdose mitigation system 100 and may provide sensor data to the processor(s) 112. For example, load sensors, position sensors, touch sensors, ultrasonic range sensors, infrared sensors, Global Positioning System (GPS) receivers, sonar, optical sensors, biosensors, force sensors, proximity sensors, Radio Frequency identification (RFID) sensors, Near Field Communication (NFC) sensors, wireless sensors, compasses, smoke sensors, light sensors, radio sensors, depth sensors (e.g., Red Green Blue plus Depth (RGB-D), lasers, structured-light, and/or a time-of-flight camera), microphones, speakers, radar, cameras (e.g., color cameras, grayscale cameras, and/or infrared cameras), and/or motion sensors (e.g., gyroscopes, accelerometers, inertial measurement units (IMU), and/or foot step or wheel odometry), among others may be used. In some embodiments, motion sensors may be used to help determine whether a user has suddenly fallen or passed out.

The overdose mitigation system 100 may include one or more alarms 108. In some embodiments, the alarm 108 is a speaker that produces a loud and audible noise after receiving an overdose indicator. The alarm 108 may also communicate (e.g., via a transmitter or the controller/app 118) with emergency personnel, care givers, or others.

Overdose mitigation system 100 may also include one or more power source(s) 120 configured to supply power to various components of the overdose mitigation system 100. Any type or combination of power source(s) 120 may be used such as, for example, one or more batteries, solar cells, or a direct, wired connection to a power source.

The user interface 122 may take various forms. In some embodiments, the user interface 122 may be a simple switch or button that the user can flip or push and indicate an input to the overdose mitigation system 100. In some embodiments, the user interface 122 may take the form of a sensor 106, such as a microphone where the user can speak and indicate an input to the overdose mitigation system 100. In some embodiments, the user interface 122 may be a graphical user interface that is integrated within the controller/app 118.

The actuator 124 may take various forms and more than one actuator 124 can be used. In some embodiments, the actuator 124 is an electroshock device that is configured to stimulate the user via shock and pain. This stimulation can be beneficial in reviving an individual from an overdose situation. In some embodiments, the actuator 124 is a speaker that plays a load noise, a transmitter that sends a help message, or a vibrating mechanism. The processor(s) 112 may actuate the actuators at periodic intervals until the overdose sensor no longer detects an overdose indicator.

In some embodiments, the overdose mitigation system 100 has a housing 126 that is an arm band device (similar to those used in a blood pressure cuff) that has an integrated wrist band pulse oximeter for its sensor 106. The arm band may be designed to part of the body (e.g., the forearm or upper arm) like a sleeve. The arm band may be held closed by Velcro straps or other means to make the arm band easy to put on and remove and facilitate the use of the system with any body type or size.

The arm band will also contain an alarm 108. If a user's oxygen saturation falls below a predetermined threshold which indicates a critical level, or an overdose indicator, the alarm 108 will produce a loud sound. If the alarm is not deactivated within a predetermined amount of time (e.g., 20 seconds, 40 seconds, or 60 seconds), a dose of an antidote such as Naxolone will be automatically delivered intramuscularly in the upper arm via a small syringe connected to the arm band device. The alarm 108 can be deactivated (thus stopping the injection) by receiving an input from the user interface 122, such as flipping a switch or pushing a button.

The injector(s) 102 may be similar to any commercially available injector or auto-injector that is designed to deliver a dose of a particular drug. In some embodiments, the injector(s) 102 may be coupled to the reservoir 103 with the antidote 104 and be placed in a non-injectable state as a default. In some embodiments, the reservoir 103 and the injector(s) 102 may be detachable from each other and from the overdose mitigation system 100 in order to be exchangeable after use or in case the antidote needs to be exchanged (e.g., if the antidote is past its expiration date and no longer approved for use).

In some embodiments, multiple injector(s) 102 may be used, and/or a single injector(s) 102 may be used that is coupled to multiple reservoirs 103, and/or the reservoir 103 may contain multiple doses of antidote, such that multiple doses of the antidote may be given. This may increase the chance of preventing death until emergency personnel or a care giver can arrive to provide further aid.

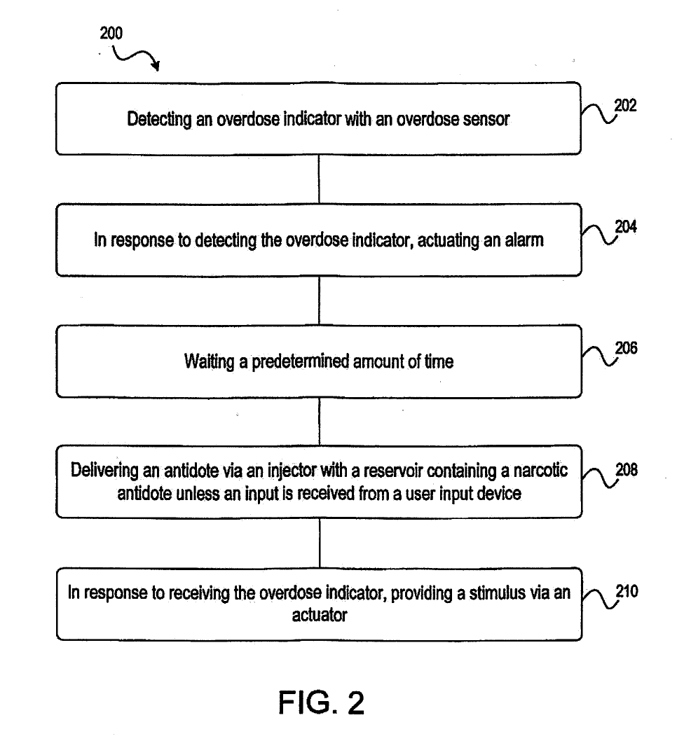

Referring now to FIG. 2, an illustrative method 200 for overdose mitigation is shown. Aspects of the method 200 may be embodied as computerized programs, routines, logic, and/or instructions executed by the overdose system 100, for example by the processor(s) 112 and one or more components of the overdose system 100, such as the injector 102. At 202, the method 200 includes detecting an overdose indicator with an overdose sensor. At 204, and in response to detecting the overdose indicator, the method 200 includes actuating an alarm. At 206, the method 200 includes waiting a predetermined amount of time which may comprise 0 seconds or 0+n seconds and may, in various embodiments, be adjusted by a user. At 208, the method 200 includes delivering an antidote via an injector with a reservoir containing a narcotic antidote, unless an input is received from a user input device. At 210, method 200 includes, in response to receiving the overdose indicator, providing a stimulus via an actuator.

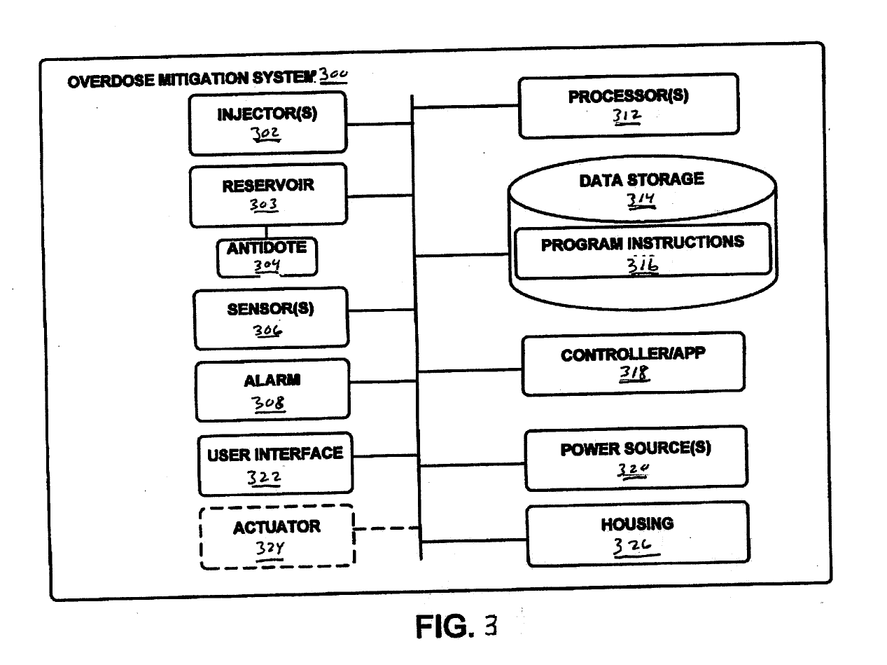

Turning now to FIGS. 3-5, certain embodiments of a system, device and method for treating nerve agent exposure are illustrated.

Nerve agent treatment system 300 includes at least one injector and may comprise two or three injectors 302, at least one reservoir, and may comprise two or three reservoirs 303 for holding an antidote, or a combination of antitodes 304 and that is coupled with and/or in fluid communication with the injector(s) 302, sensor(s) 306, an alarm 408, processor(s) 312, data storage 314, program instructions 316, a controller/app 318, power source(s) 320, a user interface 322, actuator 324, and housing 326. The nerve agent treatment system 300 is shown for illustration purposes only and may include additional components and/or have one or more components removed without departing from the scope of the disclosure. Further, the various components of the nerve agent treatment system 300 may be communicatively coupled or otherwise in communication with each other in any manner now known or later developed that enables the components to operate as a system to perform the functionality described herein.

Processor(s) 312 may be a general-purpose processor or a special purpose processor (e.g., digital signal processors, application specific integrated circuits, etc.). The processor(s) 312 can be configured to execute computer-readable program instructions 316 that are stored in the data storage 314 and are executable to cause the nerve agent treatment system 300 to perform the functions and features described herein. For instance, the program instructions 316 may be executable to provide functionality of the controller/app 318, where the controller/app 318 may be a smartphone application, or other external device, that is configured to accept a touch input to turn off the alarm and stop an injection. The controller/app 318 may be configured to communicate information as well. For example, the controller/app 318 may be configured to send a text message alert or email to emergency personnel or care givers indicating that a sensed detection and/or exposure of nerve agent has occurred and/or that a first, second and/or third injector 302 has been used.

The data storage 314 may include or take the form of one or more computer-readable storage media that can be read or accessed by processor(s) 312. The one or more computer-readable storage media can include volatile and/or non-volatile storage components, such as optical, magnetic, organic or other memory or disc storage, which can be integrated in whole or in part with processor(s) 312. In some embodiments, the data storage 314 can be implemented using a single physical device (e.g., one optical, magnetic, organic or other memory or disc storage unit), while in other embodiments, the data storage 314 can be implemented using two or more physical devices. Further, in addition to the computer-readable program instructions 116, the data storage 314 may include additional data such as diagnostic data, among other possibilities.

The nerve agent treatment system 300 may include one or more sensor(s) 306. As discussed above, one sensor 306 may comprise a sensor to detect nerve agent in the atmosphere or environment surrounding the subject wearing nerve agent treatment system 300. Such sensors are well known in the art and comprise, without limitation, sensors adapted to detect nerve agents comprising sarin, VX, tabun, soman, mustard gas or liquid, phosgene, hydrogen cyanide, whether in vapor or liquid form, to name a few. The skilled artisan will readily recognize the various types and forms of nerve agents, each of which is within the scope of the present invention. Further, the sensors 306 capable of detecting the presence of the various types and forms of nerve agents will be well known to the skilled artisan and will include, without limitation, chemical sensors including SAW chemical sensors, QCM sensors, MEMS sensors, sensors using chemicapacitor-based detection, sensors using chemiresistive-based detection including but not limited to carbon nanotubes, sensors using field-effect transistors. Each such nerve agent sensor 306 and equivalents are within the scope of the present invention and may be connected with processor 312 for provision of sensed data thereto.

Further, sensor(s) 306 may comprise a sensor that monitors the wearing subject's physical symptoms for signs and symptoms of nerve gas exposure and may include a pulse oximeter sensor to measure oxygen saturation levels. The pulse oximeter may be connected to the processor(s) 312 and configured to provide an overdose indicator should oxygen saturation levels drop below a predetermined threshold. Sensor(s) 106 may be included in nerve agent treatment system 300 and may provide sensor data to the processor(s) 312. For example, pulse rate sensors, respiratory rate sensors, load sensors, position sensors, touch sensors, ultrasonic range sensors, infrared sensors, Global Positioning System (GPS) receivers, sonar, optical sensors, biosensors, force sensors, proximity sensors, Radio Frequency identification (RFID) sensors, Near Field Communication (NFC) sensors, wireless sensors, compasses, smoke sensors, light sensors, radio sensors, depth sensors (e.g., Red Green Blue plus Depth (RGB-D), lasers, structured-light, and/or a time-of-flight camera), microphones, speakers, radar, cameras (e.g., color cameras, grayscale cameras, and/or infrared cameras), and/or motion sensors (e.g., gyroscopes, accelerometers, inertial measurement units (IMU), and/or foot step or wheel odometry), among others may be used. In some embodiments, motion sensors may be used to help determine whether a user has suddenly fallen or passed out. Each such sensor, and equivalents thereof, are within the scope of the present invention.

The nerve agent treatment system 300 may include one or more alarms 308 to annunciate the detection of nerve agent in the environment and/or symptoms of nerve agent exposure. In some embodiments, the alarm 308 is a speaker that produces a loud and audible noise after receiving indication of nerve agent detection and/or exposure. Alternatively, or in combination with a speaker-type alarm, alarm 308 may comprise a vibration and/or lighted annunciation device. The alarm 308 may also communicate (e.g., via a transmitter or the controller/app 318 using, e.g., text messages or similar communication device) with emergency personnel, care givers, or others.

Nerve agent treatment system 300 may also include one or more power source(s) 320 configured to supply power to various components of the nerve agent treatment system 300. Any type or combination of power source(s) 320 may be used such as, for example, one or more batteries, solar cells, or a direct, wired connection to a power source.

The user interface 322 may take various forms. In some embodiments, the user interface 322 may be a simple switch or button that the user can flip or push and indicate an input to the nerve agent mitigation system 300, including but not limited to a manual actuation switch or button to manually initiate the injection process. In some embodiments, the user interface 322 may take the form of a sensor 306, such as a microphone where the user can speak and indicate an input to the overdose mitigation system 300, including but not limited to voice-activated initiation of the injection process. In some embodiments, the user interface 322 may be a graphical user interface that is integrated within the controller/app 318.

The actuator 324 may take various forms, when present, and more than one actuator 324 can be used. In some embodiments, the actuator 324 is an electroshock device that is configured to stimulate the user via shock and pain. This stimulation can be beneficial in reviving an individual from an overdose situation. In some embodiments, the actuator 324 is a speaker that plays a loud noise, a transmitter that sends a help or nerve gas detection notification message via, e.g., text message, or a vibrating mechanism. The processor(s) 312 may actuate the actuators at periodic intervals until the overdose sensor no longer detects an overdose indicator.

In some embodiments, the nerve agent treatment system 300 has a housing 326 that is adapted to use with the nerve agent treatment system 300. In some cases, the housing 326 may be positioned beneath the clothes and of the wearing subject and positioned to facilitate accurate and effective injection(s). In other cases, the housing 326 may be positioned on the outside of the clothes. In some embodiments, the atmospheric or environmental sensor for detecting the presence of nerve gas may be positioned on the outside of the user's clothing and, preferably, near the user's face.

The housing may also contain an alarm 308. If a sensor detects nerve gas or related symptoms in the wearing subject, the alarm 308 will produce an annunciation, e.g., a noise, light stimulus or vibration. In some cases, if the alarm 308 is not deactivated within a predetermined amount of time (e.g., 20 seconds, 40 seconds, or 60 seconds), the antidote injection(s) will be automatically injected into the target site on the user. The alarm 308 can be deactivated (thus stopping the injection) by receiving an input from the user interface 322, such as flipping a switch or pushing a button or speaking into an interface sensor such as a microphone.

In other cases, a sensing of nerve gas or symptoms thereof by sensor(s) 306, will cause an immediate and automatic antidote injection(s) without an intervening predetermined period of time between the alarm 308 activation and injection(s).

The injector(s) 302 may be similar to any commercially available injector or auto-injector that is designed to deliver a dose of a particular element of the nerve gas antidote. In some embodiments, the injector(s) 302 may be coupled to the reservoir 303 with the antidote 304 and be placed in a non-injectable state as a default. In some embodiments, the reservoir 303 and the injector(s) 302 may be detachable from each other and from the overdose mitigation system 300 in order to be exchangeable after use or in case the antidote needs to be exchanged (e.g., if the antidote is past its expiration date and no longer approved for use).

In some embodiments, multiple injector(s) 302 may be used, and/or a single injector(s) 302 may be used that is coupled to multiple reservoirs 303, and/or the reservoir 303 may contain multiple doses of antidote, such that multiple doses of the antidote may be given. This may increase the chance of preventing death until emergency personnel or a care giver can arrive to provide further aid.

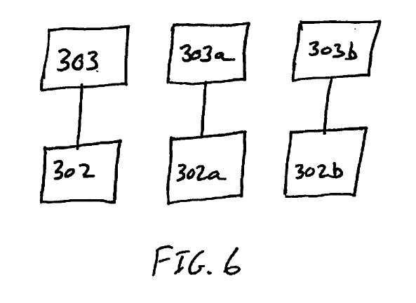

Accordingly, a first injector 302 may be coupled with a first reservoir 303 containing at least an effective dose of atropine, a second injector 302a may be coupled with a second reservoir 303a containing at least an effective dose of acetylcholinesterase reactivator, e.g., 2-PAM, and a third injector 302b may be coupled with a third reservoir 303b containing at least an effective dose of an anticonvulsant such as diazepam. An exemplary arrangement is shown in FIG. 6.

Accordingly, the processor 312 in this case may execute programmed instructions for injecting an effective dose of atropine, followed by a second injection of an effective dose of the acetylcholinesterase reactivator. If convulsions are detected by a sensor, a third injection may be automatically initiated to deliver an effective dose of diazepam. Alternatively, the first and second injectors 302, 302a may be arranged for substantially simultaneous delivery of atropine and the acetylcholinesterase reactivator from first and second reservoirs 303,303a and may, if convulsions are detected, be followed by an injection of an effective dose of diazepam with third injector 302b from reservoir 303b.

Two or more of the group consisting of atropine, the acetylcholinesterase reactivator, e.g., 2-PAM, and the anti-convulsant, e.g., diazepam, may be combined in a single reservoir. As a result, a single reservoir may comprise a mixture of each of atropine, the acetylcholinesterase reactivator and the anti-convulsant, e.g., diazepam. Alternatively, atropine and the acetylcholinesterase reactivator may be combined in a single reservoir, for injection of a combined effective dose with a single injector, with the exemplary anti-convulsant diazepam in a second reservoir for injection of an effective dose using a second injector. These injections may be achieved substantially simultaneously, e.g., in a case where convulsions are detected, or may be completed in series, with the second injection consisting of the anti-convulsant antidote only injected upon detection of convulsions. As the skilled artisan will readily recognize, any combination of the injector(s) and/or reservoir(s) are possible and within the scope of the present invention.

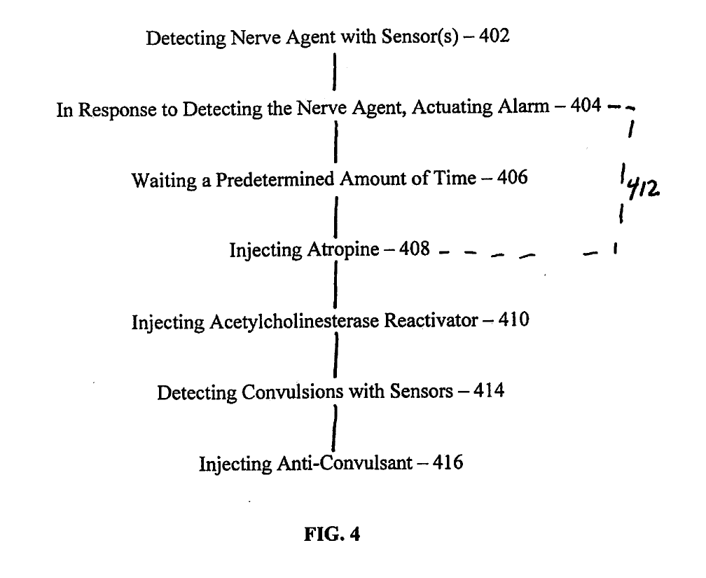

FIG. 4 thus illustrates embodiments for treating exposure of a nerve agent, beginning with detection of a nerve agent at 402. The detection may, as discussed above, be achieved by one or more sensors 306 that detect the presence of a nerve agent(s) in the immediate environment of the subject wearing the sensor and/or sense and/or detect physical symptoms in the subject as a result of nerve agent exposure. It is recognized that, since nerve agents are fast acting, that these sensor types, when both are present, may both trigger substantially simultaneously. In other cases, the environmental detection sensor may trigger first, before any physical symptoms may be detected.

Regardless of the detection mechanism, i.e., either an external atmospheric or environmental detection and/or detection of physical symptom of exposure, the system triggers an alarm at 404 which may be audible, vibratory, lighted or other annunciation mechanism to alert the wearing subject and/or companions of the presence of, or exposure to, nerve agent. 1

Once the alarm 404 is triggered, two possible alternatives are illustrated. The injection of atropine at 408 and/or the exemplary acetylcholinesterase reactivator 2-PAM at 410 may be injected as described above following the waiting period at 406. Alternatively, waiting period 406 may be omitted at 412 with the injection(s) of 408, 410 to follow immediately after alarm 404 is triggered.

Continuing with the embodiment of FIG. 4, if convulsions are detected by sensor(s) 306, at 414, then an automatic injection of anti-convulsant exemplar diazepam is executed at 416. As discussed above, these injections at 408, 410 and 416 may be combined in various ways to achieve effective treatment.

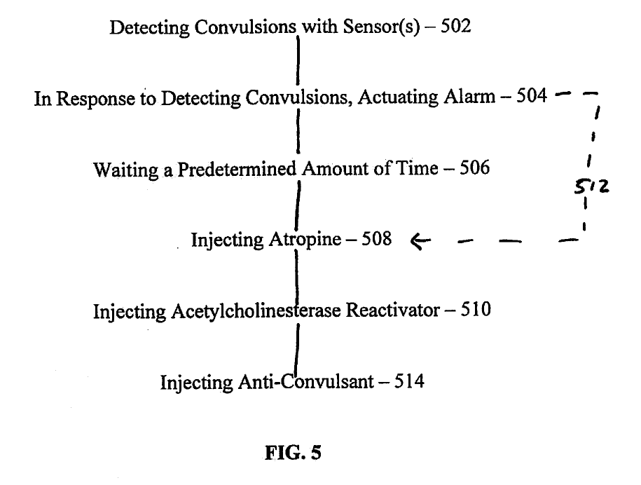

FIG. 5 illustrates a treatment for nerve agent exposure for a subject that is experiencing convulsions. In this case, the sensors 306 may have not detected any nerve agent in the environment and/or physical symptoms, with the subject moving quickly into a convulsive state which is detected at 502. Next, an alarm is triggered at 504 and an optional predetermined waiting time is provided at 506, with one embodiment incorporating the waiting time before moving to atropine injection 508 and/or the acetylcholinesterase reactivator injection 510, or the combination thereof. A second embodiment moves directly from alarm trigger 504 to atropine injection 508 and/or the acetylcholinesterase reactivator injection 510, or the combination thereof as indicated by the bypass arrow at 512. Either simultaneously, or immediately after the injection(s) of atropine injection 508 and/or the acetylcholinesterase reactivator injection 510, or the combination thereof, the diazepam injection is executed at 514. As described above, these injections of the atropine 508 and/or the acetylcholinesterase reactivator 510 and/or diazepam at 514 may be combined in any manner into one or more injections and may be executed substantially simultaneously, i.e., in parallel, or in series.

While particular aspects and embodiments are disclosed herein, other aspects and embodiments will be apparent to those skilled in the art in view of the foregoing teaching. The various aspects and embodiments disclosed herein are for illustration purposes only and are not intended to be limiting, with the true scope and spirit being indicated by the following claims.

In the foregoing description, numerous specific details, examples, and scenarios are set forth in order to provide a more thorough understanding of the present disclosure. It will be appreciated, however, that embodiments of the disclosure may be practiced without such specific details. Further, such examples and scenarios are provided for illustration only, and are not intended to limit the disclosure in any way. Those of ordinary skill in the art, with the included descriptions, should be able to implement appropriate functionality without undue experimentation.

References in the specification to “an embodiment,” etc., indicate that the embodiment described may include a particular feature, structure, or characteristic. Such phrases are not necessarily referring to the same embodiment. Further, when a particular feature, structure, or characteristic is described in connection with an embodiment, it is believed to be within the knowledge of one skilled in the art to effect such feature, structure, or characteristic in connection with other embodiments whether or not explicitly indicated.

Embodiments in accordance with the disclosure may be implemented in hardware, firmware, software, or any combination thereof. Embodiments may also be implemented as instructions stored using one or more machine-readable media which may be read and executed by one or more processors. A machine-readable medium may include any suitable form of volatile or non-volatile memory.

Modules, data structures, and the like defined herein are defined as such for ease of discussion, and are not intended to imply that any specific implementation details are required. For example, any of the described modules and/or data structures may be combined or divided in sub-modules, sub-processes or other units of computer code or data as may be required by a particular design or implementation of the computing device.

In the drawings, specific arrangements or orderings of elements may be shown for ease of description. However, the specific ordering or arrangement of such elements is not meant to imply that a particular order or sequence of processing, or separation of processes, is required in all embodiments. In general, schematic elements used to represent instruction blocks or modules may be implemented using any suitable form of machine-readable instruction, and each such instruction may be implemented using any suitable programming language, library, application programming interface (API), and/or other software development tools or frameworks. Similarly, schematic elements used to represent data or information may be implemented using any suitable electronic arrangement or data structure. Further, some connections, relationships, or associations between elements may be simplified or not shown in the drawings so as not to obscure the disclosure.

This disclosure is considered to be exemplary and not restrictive. In character, and all changes and modifications that come within the spirit of the disclosure are desired to be protected.

We claim:

1. A nerve agent treatment system for a person comprising:
at least one injector coupled with at least one reservoir, the at least one reservoir containing the group consisting of atropine, an acetylcholinesterase reactivator, and an anticonvulsant;
at least one sensor configured to detect the presence of nerve gas in the person's immediate vicinity and/or nerve gas exposure symptoms exhibited by the person; and
a processor coupled to a user interface and adapted to execute a programmable injection sequence of the at least one injector, and the at least one injector and configured to actuate the at least one injector in the programmed injection sequence in response to the detection of the presence of nerve gas and/or nerve gas exposure symptoms.

2. The nerve gas treatment system of claim 1, further comprising an alarm, wherein the alarm is actuated in response to the detection of the presence of nerve gas and/or nerve gas symptoms.

3. The nerve gas treatment system of claim 2, further comprising a predetermined amount of time immediately following actuation of the alarm and wherein the predetermined amount of time comprises zero or more seconds.

4. The nerve gas treatment system of claim 3, further comprising automatic actuation of the programmed injector sequence unless an input is received from the user interface.

5. The nerve gas treatment system of claim 1, further comprising:
a first reservoir containing atropine and in fluid communication with a first injector;
a second reservoir containing the acetylcholinesterase reactivator and in fluid communication with a second injector; and
a third reservoir containing the anti-convulsant and in fluid communication with a third injector.

6. The nerve gas treatment system of claim 1, wherein the acetylcholinesterase reactivator comprises 2-PAM and the anti-convulsant comprises diazepam.

7. The nerve gas treatment of claim 1, wherein the programmed injection sequence comprises at least an injection of an effective dose of atropine, followed by an injection of an effective dose of the acetylcholinesterase reactivator.

8. The nerve gas treatment of claim 1, wherein the at least one sensor comprises a sensor for detecting convulsions in the person and where in the programmed injection sequence comprises at least an injection of an effective dose of atropine, followed by an injection of an effective dose of the acetylcholinesterase reactivator, followed by an injection of an effective dose of the anti-convulsant.

9. The nerve gas treatment system of claim 1, wherein at least one sensor comprises a pulse oximeter that provides a nerve gas exposure symptom indicator of the person's oxygen saturation level.

10. The nerve gas treatment system of claim 1, wherein at least one sensor comprises a chemical sensor for sensing the presence of nerve gas in the vicinity of the person.

11. The nerve agent treatment system of claim 2, wherein the alarm is selected from at least one of the group consisting of: an audible alert, a visual alert, a tactile alert.

12. The overdose mitigation system of claim 1, wherein the user interface is a switch and the input is a change in a position of the switch.

13. The overdose mitigation system of claim 1 further comprising a housing enclosing at least the at least one injector and the at least one reservoir.

14. The overdose mitigation system of claim 1, wherein the reservoir is detachable from the injector.

15. The overdose mitigation system of claim 1 further comprising a power source coupled to the processor, the at least one sensor, and the at least one injector.

16. A nerve agent treatment method for a person comprising:
providing a device according to claim 2;
detecting the presence of nerve gas with a sensor and/or detecting nerve gas exposure in the person; and
in response to detecting the presence of nerve gas and/or detecting nerve gas exposure in the person:
actuating an alarm; and
automatically injecting a nerve gas antidote into the person according to a programmed injection sequence.

17. The nerve agent treatment method of claim 16, wherein the nerve gas antidote comprises atropine and 2-PAM and wherein the programmed injection sequence comprises injecting an effective dose of atropine followed by injecting an effective dose of 2-PAM.

18. The nerve agent treatment method of claim 15, further comprising:
detecting convulsions in the person and wherein the nerve gas antidote comprises an anti-convulsant.

19. The nerve agent treatment method of claim 18, wherein the anti-convulsant comprises diazepam.

20. The nerve agent treatment method of claim 17, further comprising detecting convulsions in the person and wherein the nerve gas antidote further comprises diazepam and wherein the programmed injection sequence further comprises injecting an effective dose of diazepam after the injecting of an effective dose of 2-PAM.

21. The nerve agent treatment method of claim 16, further comprising sending at least one alert message using text messaging or radio messaging after at least one of the group consisting of: detecting the presence of nerve agent, detecting symptoms in the person, actuating the alarm, and injecting a nerve gas antidote into the person according to the programmed injection sequence.
